(19) United States
(12) Patent Application Publication
(10) Pub. No.: US 2017/0079692 A1
(43) Pub. Date: Mar. 23, 2017

(54) Osteosynthesis Assembly Formed by a Plate and at Least One Screw

(71) Applicant: KISCO INTERNATIONAL, Saint Priest (FR)

(72) Inventors: Virgile CIRIER, Caluire et Cuire (FR); Jean-Claude HUOT, Pau (FR); Lionel SIMON, Solaize (FR); Jean-Marc VITAL, Bordeaux (FR)

(21) Appl. No.: 15/311,388

(22) PCT Filed: May 7, 2015
(86) PCT No.: PCT/EP2015/060035

§ 371 Date: Nov. 15, 2016

(30) Foreign Application Priority Data
May 16, 2014 (FR) 1454402

(51) Int. Cl. A61B 17/70; A61B 17/86; A61B 17/80

(52) U.S. Cl. CPC A61B 17/7059; A61B 17/8052; A61B 17/8605; A61B 17/863

(57) ABSTRACT

The invention relates to an osteosynthesis assembly formed by a plate and at least one screw provided with a head, designed to maintain said plate by crossing through an orifice of said plate to fasten it to a bone, said plate having as many orifices (2) as there are screws, characterized in that:

at least one of said orifices of said plate has, over at least a portion of its periphery, a metal, ceramic or polymer collar, at which the nominal diameter of the orifice is reduced to a value below said nominal diameter;

said screw designed to cross through said orifice has a conical or curved under-head threading;

the collar and the thread bottom of the under-head threading have a tightening of at least 0.01 mm if the collar is made from metal or ceramic and at least 0.05 mm if the collar is made from polymer, so as to create a hard spot toward the end of the placement of the screw, said hard spot remaining after the screw has been placed.

The present invention relates to plates designed for the assembly of two medical devices, including a screw, designed to be implanted on a bone of a patient during a surgical procedure. It more specifically relates to the bone screw-plate assemblies that are used in surgery of the spinal column to stabilize the vertebrae, in particular during arthrodesis of the cervical vertebrae.

When a plate made from a biocompatible metal material (stainless steel, titanium, etc.) or a polymer is used to stabilize two (or more) vertebrae relative to one another, the plate is fastened using screws that may be self-tapping and/or self-piercing, generally in pairs (two screws per vertebral level) and crossing through corresponding orifices arranged in the plate. It is of course important for these screws not to be able to gradually back out of their implantation location by withdrawing over the course of the use of the plate, and various anti-withdrawal devices have been designed to that end.

One commonly used device is the installation on the surface of the plate of an element that covers the head of one or more of the screws, and which itself is secured to the plate. This device has the drawback of requiring additional elements on top of the plate and screws, therefore extending the placement time of the device and making it more complex, as well as making its removal more complex when it is necessary to replace an element of the device. Furthermore, the thickness of the assembly may be increased as a result, which is a drawback, in particular in the cervical region.

Other devices exist, in which an additional part is unnecessary, and where the screw is secured to the plate by a deformable edge of said orifice which, during passage of the screw, assumes a shape such that spontaneous removal of the screw becomes impossible. However, this deformation is irreversible and makes the plate unusable as soon as just one screw needs to be replaced.

Another functionality that must preferably be present on such plates is the possibility of easily giving the longitudinal axis of each screw a selected orientation, which may be different from an orientation simply combined with that of the longitudinal axis of the corresponding orifice and/or that is perpendicular to the general orientation of the plate if the latter is substantially planar. In other words, the screw must be able to be of the multi-axial type.

The aim of the invention is to propose a configuration of the screw/plate assembly that makes it possible to propose, in a satisfactory manner, all of the aforementioned functionalities, namely a plate that may have a small thickness over its entire expanse once fastened on the vertebrae, the possibility of multi-axial screws and the impossibility of spontaneous gradual backing out of the screws.

To that end, the invention relates to an osteosynthesis assembly formed by a plate and at least one screw having a thread bottom diameter provided with a head, designed to maintain said plate by crossing through an orifice of said plate to fasten it to a bone, said plate having as many orifices as there are screws, characterized in that:

at least one of said orifices of said plate has, over at least a portion of its periphery, a metal, ceramic or polymer collar, at which the nominal diameter of the orifice is reduced to a value below said nominal diameter;

said screw designed to cross through said orifice has a conical or curved under-head thread;

the collar and the thread bottom of the threading below the head have, in at least one section of the screw, a tightening of at least 0.01 mm if the collar is made from metal or ceramic and at least 0.05 mm if the collar is made from polymer, so as to create a hard spot toward the end of the placement of the screw, said hard spot remaining after the screw has been placed.

Said screw may have a smooth part between the under-head threading and the head, the diameter of said smooth part being smaller than the diameter of the collar and the length of said smooth part being greater than the height of the collar.

Said collar may have a variable width and/or thickness along its perimeter.

Said collar may have notches and/or indentations and/or localized thinner areas.

The under-head threading of said screw may have an even number of threads.

The localization of the under-head threading allows the latter to be anchored in the cortical bone when the screw is in place.

The relative dimensions of the various parts of the screw and the collar can allow an incline of the screw relative to the longitudinal axis of the orifice of up to ±15°.

Said collar may be attached on the plate.

As will have been understood, the invention is based on the presence of:
On the one hand, very slightly deformable collars present on the inner edge of, preferably, each orifice of the plate in which a screw is inserted;
On the other hand, a conical or curved thread arranged on the corresponding screw and placed just below the head of the screw, or only separated from the head of the screw by a short smooth portion authorizing an incline of the longitudinal axis of the screw relative to the axis of the orifice in which it is inserted;
And relative dimensions of the collar and bottom of the thread under the screw head that create, during the screwing and unscrewing by the surgeon, a “hard spot” that requires that part of the screw to pass forcibly past the collar, which, to that end, deforms slightly and temporarily, which causes spontaneous rising of the screw during use of the plate to be prevented; indeed, since the collar and the under-head threading have not been significantly damaged during tightening, the conditions for creating the hard spot remain at the end of the screwing; the crossing of that hard spot by the under-head thread in the opposite direction is therefore only possible if a deliberate unscrewing force is applied to the screw, substantially greater than the force that is naturally exerted on the collar upon the beginning of spontaneous rising of the screw.

In order to create this hard spot, while allowing it to be crossed during screwing without significant damage to the collar and/or the screw so as to allow it to remain after complete screwing, the inner diameter of the collar and the maximum diameter of the bottom of the under-head thread have a tightening of at least 0.01 mm when the collar is made from metal or ceramic, and at least 0.05 mm when the collar is made from polymer. Additionally, the outer diameter of the under-head thread must be at least 0.05 mm greater than the inner diameter of the collar when the latter is made from metal, and at least 0.1 mm when the latter is made from polymer. It is considered that one must not go below these values to create a sufficiently effective hard spot.

Conversely, it is also necessary for this tightening not to be excessive, in order to avoid deformation by irreversible crushing of the screw and/or the collar to the point that after screwing, the opening of the collar and the under-head thread would have substantially modified dimensions, such that, during rising of the screw, the latter would no longer encounter enough resistance from the collar. Maximum tightening of approximately 1 mm would be advisable in many cases, but the precise maximum value of the allowable tightening depends greatly on the natures of the respective materials of the screw and the collar, as well as the shape of the collar that contributes to determining its reversible or irreversible deformation capacity during the passage by the hard spot during screwing. It will be easy to determine the maximum allowable tightening experimentally to that end, for given collar morphologies and screw and collar materials.

It must be understood that “tightening” refers to the difference between the minimum opening of the collar and the maximum diameter of the screw at the thread bottom of the under-head threading, the latter being greater than it by a value equal to said tightening.

Of course, in the most general case where the collar is arranged directly on the plate itself, the material of the collar and the material of the plate will be the same. However, it is possible to consider providing different materials for both if the collar is arranged using a separate element attached on the plate (for example, through a lateral insertion of the element through a slot crossing through the plate and emerging in the orifice), as will be possible to see. This possibility of providing a collar not integral with the plate provides greater flexibility to the surgeon choosing the elements of the assembly and the dimensions. Care must, however, be taken in making sure that this technological choice does not lead to increasing the thickness of the plate in a manner that would make it uninteresting to use in the spinal region where it is designed to be implanted, in particular in the cervical region.

In any case, the diameter of the bottom of the thread 10 outside the zone bearing the under-head thread is smaller than the inner diameter of the collar, so as to allow the screw to pass the collar without difficulty until the latter encounters the under-head thread.

By acting on the geometry of the plate, it is possible, in a known manner, to provide that the screw penetrates the bone in a direction having an angulation with the longitudinal axis of the orifice. In other words, a polyaxial configuration of the screw/plate assembly is possible by using the invention. To that end, it is possible to provide, between the under-head threading and the head, a zone with no threading and the diameter of which is smaller than that of the opening of the collar. In this way, the angulation of the screw relative to the axis of the orifice is still possible for the final tightening, and micro-movements of the screw are possible after tightening.

The dimension of the hard spot can be better controlled if the screw has an even number of under-head threads, since an even number makes it possible to control the diameter obtained by two opposite threads. Such threads make it possible to drive the screw through the plate by the tightening torque only because they become engaged on the collar like a screw in a nut. The passage of the hard spot during screwing is therefore done without it being necessary to exert any force on the bone. This feature also makes it possible to exert symmetrical pressure between the screw and the collar relative to the axis of the screw, which preserves the bone from additional stresses during screwing during the passage of the hard spot, since the progression axis of the screw is, as a result, invariable, in particular in the case where the axis of the screw is inclined relative to the longitudinal axis of the orifice.

In one example embodiment, the collar travels the entire perimeter of the orifice of the plate, with a constant width and thickness. However, it is also possible only to arrange this collar on part of said perimeter, i.e., to provide notches, indentations or localized thinner portions on the collar in terms of width and/or thickness. Combined with the choice of materials and dimensions of the parts, the geometry of these notches/indentations/thinner portions provides additional freedom to determine the force necessary to cause the screw to pass the hard spot. It is possible, for example, to provide at least two notches each 0.5 mm wide. Preferably, in general, at least half of the perimeter of the orifice will have a collar sized so as to constitute a hard spot under the conditions according to the invention.

It is possible to provide for situating the conical or curved threading below the head at a distance from the head and over a length such that, after having passed the collar, this threading penetrates the cortical bone. The dimensional characteristics of the conical or curved threading are then particularly suitable for that purpose.

Of course, it is preferable that if the plate has several orifices, each of them is provided for the collar according to the invention and is crossed through by a screw also designed according to the invention. However, it remains within the spirit of the invention to provide that only some of the orifices are provided with such a collar or such a screw.

The invention will be better understood upon reading the following description, given in reference to the following appended figures:

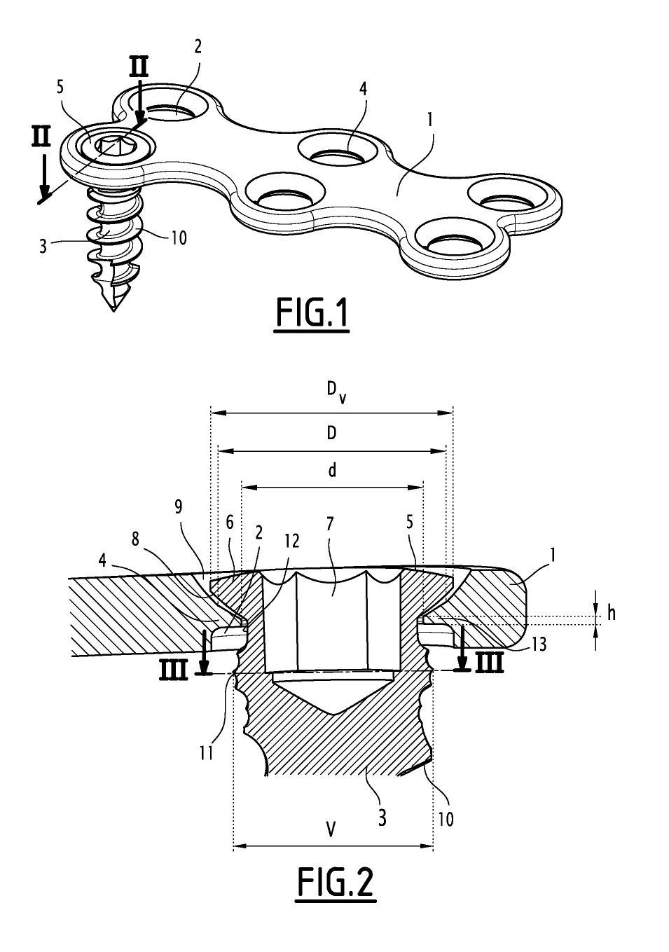

FIG. 1, which shows an example of a cervical plate according to the invention, with an associated screw;

FIG. 2, which shows, in longitudinal section along II-II, a portion of that plate, i.e., one of the orifices in which a screw is designed to be inserted, as well as the upper part of the corresponding screw;

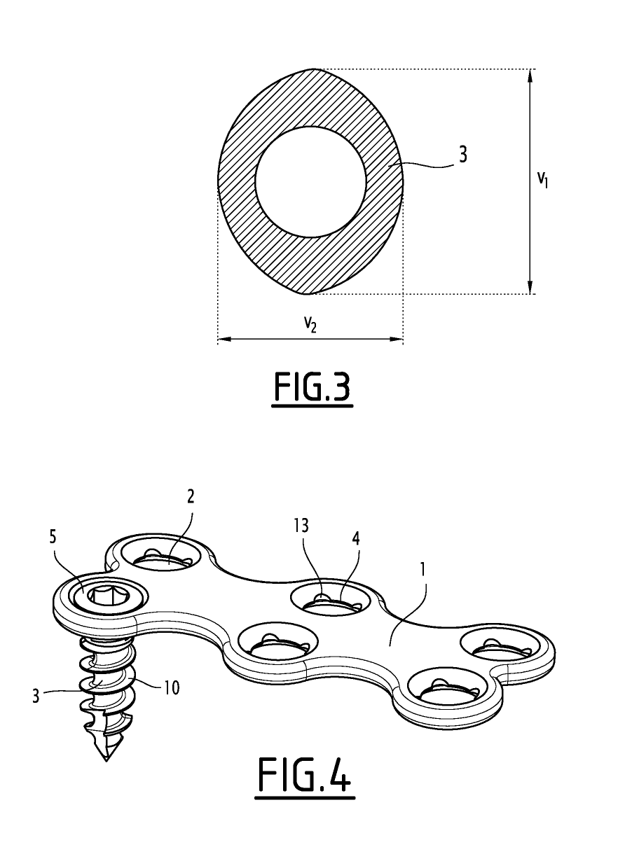

FIG. 3, which shows a cross-section along III-III of said screw at the conical and/or curved threading, which, according to the invention, is formed therein below the head;

FIG. 4, which shows another example of a cervical plate according to the invention, with an associated screw, the collars of the plate being provided with notches.

The plate 1 according to the invention is, in the non-limiting example shown in FIG. 1, of the type designed to extend over three cervical vertebrae. It therefore has three pairs of orifices 2, each being designed to be crossed through by a screw 3 (only one of which is shown in FIG. 1), which must penetrate a vertebra (in the case at hand, a cervical vertebra).

As better shown in FIG. 2, the plate 1 includes, according to the invention, over at least one of the parts of the periphery of each of its orifices 2, a collar 4 that gradually reduces, over its thickness h, the nominal diameter D of the orifice 2 to a minimum value d.

The screw 3 according to the invention has a head 5 with a traditional configuration, in that it has, on its upper face 6 with diameter Dv, a housing 7 with a perimeter that is for example hexagonal for the engagement of an instrument, such as a screwdriver, and in the lower face has a step 8 designed to cooperate with a corresponding step 9 of the orifice 2 of the plate 1. Over most of its length, the screw 3 has a threading 10 for its anchoring in the bone of the corresponding vertebra. The thread bottom diameter of that threading 10 is smaller than the nominal diameter D of the orifice 2 and, in the illustrated example, also the diameter d defined by the collar 4. Thus, the screw 3 can cross through the collar 4 of the orifice 2 without difficulty.

According to the invention, below the head 5 of the screw 3, there is another threading 11 with a conical or curved shape, the thread bottom diameter v₁ has, at least in one section of the screw, a value greater than d so as to create a hard spot when the under-head threading 11 comes into contact with the collar 4. The diameter denoted v₂ in FIG. 3, smaller than v₁, corresponds to the element of the tool that creates a smaller-diameter zone of the under-head threading 11.

As stated, (v₁-d) is greater than or equal to 0.01 mm when the collar 4 is made from metal or ceramic and greater than or equal to 0.05 mm when the collar 4 is made from polymer.

V refers to the maximum diameter of the under-head threading 11. V defines an axial stop of the screw on the collar with diameter d so as to prevent untimely backing out of the screw. The under-head threading 11 engages in the collar like a screw in a nut during the progression of the screw 3, and owing to the tightening or loosening torque, it makes it possible to drive the screw in or outside the plate without stress on the bone. However, it cannot cross the hard spot unless a sufficient torque is deliberately applied to the screw 3 by the surgeon, such that spontaneous withdrawal of the screw 3 causing unwanted passage by the hard spot, therefore spontaneous withdrawal of the screw outside the bone, is not possible.

The under-head thread 11 has, in the illustrated example and as is preferable, an even number of threads, in the case at hand, two, for the reasons previously stated.

The under-head threading 11 is, preferably and as shown, present in a location that makes it possible to find it in the cortical bone of the vertebra after tightening of the screw 3. To that end, an increase in the diameter of the thread bottom favors the bone compression, and an increase in the number of threads favors the attachment of the screw in the cortical bone.

The pitch of the under-head threading 11 is preferably equal to or close to that of the bone threading 10, and thus exerts little axial force on the bone upon passing by the hard spot. For example, the two pitches only differ by 0.2 mm.

The rest of the threading of the screw 4 is suitable for being ideally fastened in the bone, as is traditionally the case.

According to one preferred alternative of the invention, between the under-head threading 11 and the head 5 of the screw 3 is a smooth part 12, the length of which is preferably greater than the height h of the collar 4, for example comprised between 0.3 and 0.5 mm, and with a diameter smaller than d. It makes it possible to release the thread 11 from the plate and thus to ensure contact between the screw head and the plate by the incline selected by the surgeon, in the incline interval defined by its diameter, for example a maximum of up to ±15° relative to the longitudinal axis of the hole of the plate. Thus, the tightening of the plate in the bone using the screw is done without force between the plate and the screw other than the force desired in the axis of the screw.

As shown in FIG. 4, in the illustrated example, it is possible to provide indentations 13 on the collar 4, which here assume a half-circle shape. It is preferable for these notches not to be diametrically opposite so as not to alter the operation of the hard spot. For example, it is possible to provide three notches distributed 120° apart from one another.

As an example, it is possible to provide a titanium plate 1 and a screw 3 also made from titanium, the plate 1 having orifices 2 with a diameter D of 5 mm, collars with a diameter d of 3.9 mm, and the screw 3 having a maximum head diameter Dv of 5.3 mm and an under-head threading 11 with a maximum diameter V of 4.3 mm and thread bottom diameter v₁ of 4 m.

As shown in the illustrated example, it is possible, by correctly sizing the different parts of the plate and the screw, to have the head of the screw not protrude past the upper face of the plate 1. This makes the invention particularly well-suited to cervical plates, the total thickness of which must be as small as possible so that they can be operational in their environment.

The invention has been described in reference to a cervical plate-screw assembly, but the invention can of course apply to all types of osteosynthesis devices including a plate crossed through by one or more screws, for which it is desirable to prevent the ability to gradually leave their housing. The invention also has the noteworthy advantage of not requiring any additional part to that end that could extend the placement time of the device and be incorrectly placed during that placement, or be lost, or that would give the assembly that it forms with the plate and the screws a thickness exceeding that which the plate and the heads of the screws alone require.

1. An osteosynthesis assembly comprising:
a plate that comprises at least one orifice having a periphery and a nominal diameter;
a metal, ceramic, or polymer collar over a least a portion of the periphery of the orifice of the plate, wherein the collar has a diameter, d, that is less than the nominal diameter of the orifice, and wherein the collar is integral with or attached to the plate;
and at least one screw having a conical or curved under-head threading that has a thread bottom with a diameter, v₁, and a head, wherein each screw is designed to cross through one orifice of said plate to fasten the plate to a bone;
wherein the collar and the thread bottom of the under-head threading have, in at least one section of the screw, a tightening, v₁-d, of at least 0.01 mm if the collar is made from metal or ceramic and at least 0.05 mm if the collar is made from polymer, so as to create a hard spot toward the end of the placement of the screw, said hard spot remaining after the screw has been placed.

2. The assembly according to claim 1, wherein the collar also has a height, and wherein the said screw has a smooth part between the under-head threading and the head, wherein the smooth part has a diameter that is smaller than the diameter of the collar and a length that is greater than the height of the collar.

3. The assembly according to claim 1, wherein said collar has a variable width and/or thickness along its perimeter.

4. The assembly according to claim 3, wherein that said collar has notches and/or indentations and/or localized thinner areas.

5. The assembly according to claim 1, wherein the under-head threading of said screw has an even number of threads.

6. The assembly according to claim 1, wherein the location of the under-head threading allows the it to be anchored in a cortical bone when the screw has been placed.

7. The assembly according to claim 2, wherein the relative dimensions of the various parts of the screw and the collar allow an incline of the screw relative to a longitudinal axis of the orifice of up to ±15°.

8. The assembly according to claim 1, wherein said collar is attached to the plate.
